(19) United States
(12) Patent Application Publication
(10) Pub. No.: US 2022/0054139 A1
(43) Pub. Date: Feb. 24, 2022

(54) EMBOLIC BASKET, PARTICLES, AND RELATED METHODS

(71) Applicants: Michael Hallisey, Old Saybrook, CT (US); Merit Medical Systems, Inc., South Jordan, UT (US)

(72) Inventors: Michael Hallisey, Old Saybrook, CT (US); Jim Mottola, West Jordan, UT (US); Steven Weir, Sandy, UT (US)

(21) Appl. No.: 17/404,027

(22) Filed: Aug. 17, 2021

Related U.S. Application Data
(60) Provisional application No. 63/066,816, filed on Aug. 18, 2020.

(51) Int. Cl. A61B 17/12; A61B 17/122

(52) U.S. Cl. CPC A61B 17/12113; A61B 17/12145; A61B 17/1215; A61B 2017/00004; A61B 17/122; A61B 2017/1205; A61B 17/12181

(57) ABSTRACT

Devices used to restrict flow within a blood vessel are disclosed. Devices within the scope of this disclosure include a braided lattice of nitinol wires that form self-expanding enclosures of an embolic structure. The devices may further include embolic particles disposed within the enclosures. Methods of deploying the devices with the embolic particles are disclosed. Methods of manufacturing the devices with the embolic particles disposed within the enclosures are disclosed.

### RELATED APPLICATION

This application claims priority to U.S. Provisional Application No. 63/066,816, filed on Aug. 18, 2020, and titled “EMBOLIC BASKET, PARTICLES, AND RELATED METHODS” which is hereby incorporated by reference in its entirety.

### TECHNICAL FIELD

The present disclosure generally relates to intravascular devices for treating certain medical conditions, including use of low profile intravascular occlusion devices to treat vascular defects and/or to prevent blood flow within a blood vessel.

### BRIEF DESCRIPTION OF THE DRAWINGS

The embodiments disclosed herein will become more fully apparent from the following description and appended claims, taken in conjunction with the accompanying drawings. These drawings depict only typical embodiments, which will be described with additional specificity and detail through use of the accompanying drawings in which:

FIG. 1 is an image of an embodiment of an embolization device in an expanded state with embolic particles disposed within enclosures of an embolic structure.

FIG. 2 is an image of the embolic particle of the embolization device of FIG. 1.

FIG. 3 is an image of the embolic particle of FIG. 1 disposed within a partially expanded enclosure of FIG. 1.

FIG. 4 is a magnified view of the embolic particle of FIG. 1 disposed within a partially expanded enclosure of FIG. 1.

FIG. 5 is an image of the embolization device of FIG. 1 deployed within a blood vessel.

### DETAILED DESCRIPTION

A wide variety of intravascular devices are used in various medical procedures. For example, embolization devices may be used to treat arterial-venous malformations, aneurysms, and other vascular defects, or to prevent blood flow to tumors or other portions of the body.

In some instances, an embolization device includes an embolic structure comprising a plurality of enclosures or baskets. Enclosures within the scope of this disclosure include baskets of a woven lattice or matrix, including embodiments formed of nitinol wires. The plurality of enclosures may be coupled together and releasably coupled to a guidewire. The enclosures can be crimped or constrained to a small diameter and disposed within a delivery catheter for deployment into a blood vessel. In some embodiments, in a fully expanded configuration, the enclosures have a disk shape. In a partially expanded state, the enclosures may be elongate, spherical, ovoid, cylindrical, or other shapes. The enclosures may be configured to restrict blood flow through the blood vessel when deployed within a blood vessel. When deployed the enclosure may be fully or partially expanded, including instances where the degree of expansion is controlled by interaction between the vessel wall and the enclosure.

In some embodiments within the scope of this disclosure, an embolic particle may be disposed within one or more enclosures and deployed with the enclosures. When deployed, the embolic particle may increase the restriction of blood flow through the blood vessel. Stated another way, a plurality of enclosures wherein one or more enclosures contains an embolic particle may reduce flow through a vessel more than the plurality of enclosures alone. For example, in one ex vivo experiment, a 25% drop in blood flow was seen at 100 mmHG with an embolization device with embolic particles as compared to an embolization device without embolic particles.

Embolization devices within the scope of this disclosure can be manufactured by weaving filaments or wires to create a lattice or basket defining the enclosure. Filaments within the scope of this disclosure include metals and polymers, including superelastic materials. For example, nitinol wires may be used to form the embolic structure of the enclosures. In some embodiments, a continuous weave of filaments may be used to form a plurality of enclosures with necked down middle portions disposed between the enclosures. During manufacturing, one or more embolic particles can be disposed within a needle and injected into the enclosures, with the enclosures in a partially expanded state. The embolic structure, with particles inside, may be crimped to a small diameter to fit within a delivery catheter.

An embolization device may be used in procedures to occlude vascular structures such as blood vessels. The embolization device can be deployed into a blood vessel by positioning a guide catheter at a desired deployment location for the embolization device, inserting the delivery catheter into the guide catheter, deploying the embolic structure with the embolic particle disposed within the embolic structure into the blood vessel, and releasing the embolic structure from a guidewire. Once deployed, the embolic structure can self-expand until it contacts the vessel wall. When expanded, the woven lattice of the embolic structure and the embolic particle may restrict blood flow through the blood vessel.

Embodiments may be understood by reference to the drawings. It will be readily understood by one of ordinary skill in the art having the benefit of this disclosure that the components of the embodiments, as generally described and illustrated in the figures herein, could be arranged and designed in a wide variety of different configurations. Thus, the following more detailed description of various embodiments, as represented in the figures, is not intended to limit the scope of the disclosure, but is merely representative of various embodiments. While the various aspects of the embodiments are presented in drawings or figures, these are not necessarily drawn to scale unless specifically indicated.

It will be appreciated that various features are sometimes grouped together in a single embodiment, figure, or description thereof for the purpose of streamlining the disclosure. Many of these features may be used alone and/or in combination with one another.

The phrase “coupled to” refers to any form of interaction between two or more entities, including mechanical, electrical, magnetic, electromagnetic, fluid, and thermal interaction. Two components may be coupled to each other even though they are not in direct contact with each other. For example, two components may be coupled to each other through an intermediate component.

The directional terms “distal” and “proximal” are given their ordinary meaning in the art. That is, the distal end of a medical device means the end of the device furthest from the practitioner during use. The proximal end refers to the opposite end, or the end nearest the practitioner during use.

“Fluid” is used in its broadest sense, to refer to any fluid, including both liquids and gases as well as solutions, compounds, suspensions, etc., that generally behaves as a fluid.

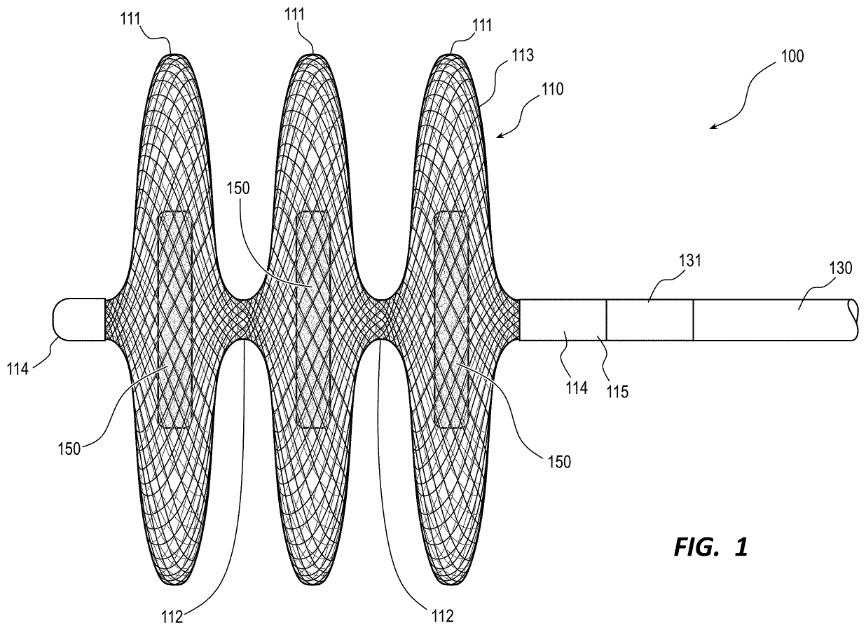

FIGS. 1-5 illustrate different views of an embolic device and related components. In certain views each device may be coupled to, or shown with, additional components not included in every view. Further, in some views only selected components are illustrated, to provide detail into the relationship of the components. Some components may be shown in multiple views but not discussed in connection with every view. Disclosure provided in connection with any figure is relevant and applicable to disclosure provided in connection with any other figure or embodiment.

FIG. 1 depicts one embodiment of an embolization device 100 in a pre-load or expanded state. In the illustrated embodiment, the embolization device 100 includes an embolic structure 110 of three enclosures or baskets 111 with necked down middle portions 112 disposed between the enclosures. In another embodiment, the embolic structure 110 may include a single enclosure. In yet another embodiment, the embolic structure 110 may include two enclosures with a necked down middle portion disposed between the two enclosures. Embodiments with more than three enclosures, including embodiments with four, five, six, or more enclosures, are likewise within the scope of this disclosure. In the illustrated embodiment, in the pre-load or expanded state, the enclosures 111 have a disk shape. Embodiments where the expanded shape is spherical, ovoid, cylindrical, or any other shape are likewise within the scope of this disclosure.

In the illustrated embodiment, the embolic structure 110 includes a braided lattice or matrix of wires 113. In some embodiments, the wires 113 can be formed of any suitable material that exhibits a shape memory effect. For example, the wires 113 may be formed of shape memory metals such as nickel-titanium alloy, copper-zinc-aluminum alloy, iron-manganese-silicon, and copper-aluminum-nickel alloy, or from shape memory polymers such as polytetrafluoroethylene (PTFE), polylactide (PLA), and ethylene-vinyl acetate (EVA). In certain embodiments, the wires 113 are formed of nitinol. Other shape memory metals and polymers are contemplated within the scope of this disclosure. The ends of the wires 113 can be restrained by clamps 114 to prevent fraying of the braid. The embolic structure 110 can be releasably coupled to a placement wire 130 for deployment. For example, in the illustrated embodiment the embolic structure 110 includes a threaded clamp 115 that can be threadingly coupled to a threaded end 131 of the placement wire 130. When deployed, the embolic structure 110 can be held in place relative to the placement wire 130 when the embolic structure 110 engages with the vessel wall and the placement wire 130 can be rotated to release the placement wire 130 from the embolic structure 110. Other mechanisms for release and deployment are also within the scope of this disclosure including, hooks, collets, loops, snares, and so forth.

FIG. 1 also depicts an embolic particle 150 disposed within each of the enclosures 111 of the embolization device 100. The embolic particle 150 can be configured to promote thrombosis or clotting of blood. In other embodiments, two or more embolic particles 150 may be disposed within each of the enclosures 111. In another embodiment, one, two, three, or more embolic particles 150 may be disposed within each enclosure 111. Furthermore, embodiments wherein the embolic particles 150 are disposed within only a subset of the total number of the enclosures 111, including embodiments wherein any number of the embolic particles 150 may be disposed within any number of the enclosures 111, are within the scope of this disclosure.

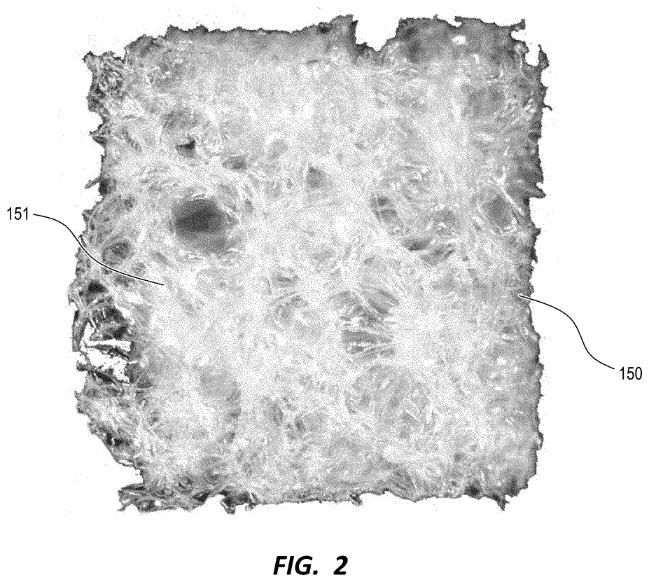

FIG. 2 illustrates the embolic particle 150 prior to disposing of the embolic particle into the enclosure 111 of the embolic structure 110. As shown, the embolic particle 150 can be a cube of gelatin material. The embolic particle 150 may have a generally defined shape when dry, but may transition to comprise a material similar to a viscous fluid when hydrated. As shown in the figure, the embolic particle 150 may comprise pores, cells, or openings 151 when in the dry configuration, including embodiments formed of gelatin material in the form of a matrix or a foam. The embolic particle 150 may not dissolve in water or blood, but may soften and have little defined shape when hydrated. In other words, embodiments wherein the embolic particle 150 has a generally defined shape when dry and an amorphous shape when hydrated are within the scope of this disclosure. Additionally, the embolic particle may be bioabsorbable within the body, including materials that can be absorbed by the body over a period of weeks or months. Again, when hydrated, the embolic particle 150 may have an amorphous shape that may return to the pre-hydrated defined shape, or partially return to the defined shape, when dried. The embolic particle 150 can have cube dimensions that range from about 1 millimeter to about 10 millimeters, including from about 2.5 millimeters to about 5 millimeters. In other embodiments, the embolic particle 150 can be formed of any suitable material, such as collagen, polyvinyl alcohol, etc. In some embodiments, the embolic particle 150 may include a thrombogenic agent, such as thrombin, configured to promote thrombus formation adjacent the embolic particle 150. In another embodiment, the embolic particle 150 can have any suitable form. For example, the embolic particle 150 can be a cylinder, a ball, an amorphous form, an irregular shape, etc. Embodiments wherein the embolic particle 150 is formed of natural materials, synthetic materials, porous materials, bioabsorbable materials, biostable material, and other materials are all within the scope of this disclosure.

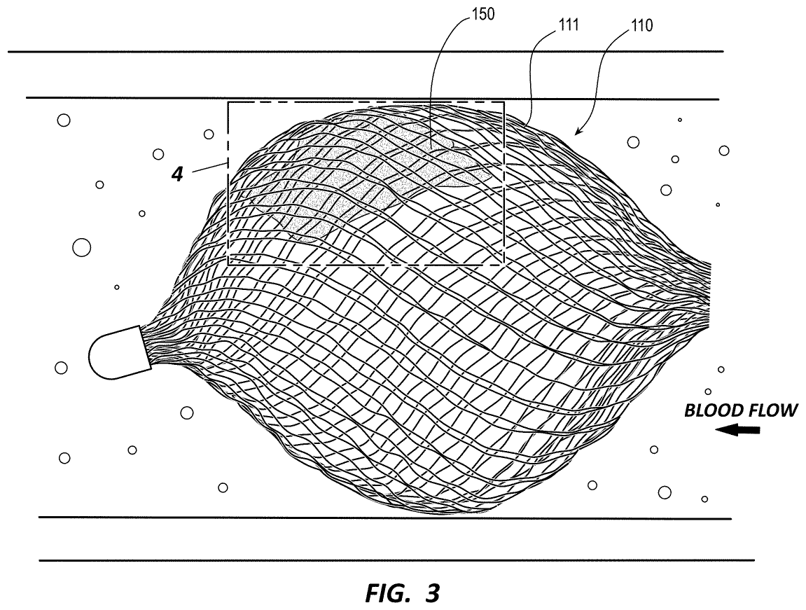

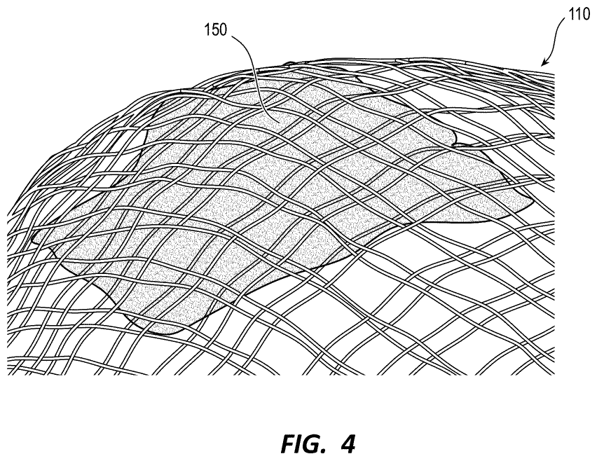

The embolic particle 150 may be placed within an enclosure by placing the dry embolic particle 150 within a hub of a needle or blunt cannula. A diameter of the needle or blunt cannula may range from about 16 gauge to about 23 gauge, including from about 18 gauge to about 22 gauge, and can be about 21 gauge. A fluid dispensing device (e.g., syringe or high-pressure syringe) containing a fluid such as water or saline may be coupled to the hub. The distal end of the needle may be disposed through an opening of the lattice of a partially expanded enclosure 111. The syringe can be pressurized, causing the embolic particle 150 to be hydrated within the hub, taking on the characteristics of a viscous material and subsequently injected into the enclosure 111 of the embolic structure 110. The embolic particle 150 can be dried within the enclosure 111. The configurations shown in FIGS. 3 and 4 include the embolic particle 150 that has been injected into the enclosure 111 and dried.

In a certain embodiment, the embolic structure 110 and the embolic particles 150 may be provided to a user in an expanded state such as shown in FIG. 1. When preparing the embolic structure 110 for use, the user may hydrate the embolic particles 150 with water or saline (thus softening the embolic particles 150) and transition the embolic structure 110 and the embolic particles 150 into a constrained state by pulling or otherwise disposing the embolic structure 110 and the embolic particles 150 into a delivery catheter to reduce a diameter of the embolic structure 110 and the embolic particles 150.

In another embodiment, the embolic structure 110 and the embolic particles 150 may be provided to a user in the constrained state where the embolic structure 110 and the embolic particles 150 are crimped to a small diameter and disposed within the delivery catheter.

The embolization device 100 can be deployed within a blood vessel by advancing the delivery catheter containing the embolic structure 110 and the embolic particles 150 to a treatment location in the body and deploying the embolic structure 110 and the embolic particles 150. In some embodiments, this may include loading the delivery catheter containing the constrained embolic structure 110 and the embolic particles 150 into a guide catheter and advancing the delivery catheter to a distal end of the guide catheter. The delivery catheter may be displaced proximally relative to the embolic structure 110 and the embolic particles 150 such that the embolic structure 110 and the embolic particles 150 are disposed within the blood vessel. The embolic structure 110 may be configured to self-expand as it is deployed within the blood vessel.

During such deployments, the embolic structure 110 and the embolic particles 150 may be disposed within the blood vessel together or simultaneously such that a secondary deployment or injection is not needed. That is, placing the embolic structure 110 including the enclosures 111 and the embolic particles 150 within a blood vessel may simultaneously place the enclosures 111 and the embolic particles 150 within the blood vessel. Similarly, a single deployment action, such as retracting a delivery catheter, may thus deliver both the enclosures 111 and the embolic particles 150 into the blood vessel in a deployed configuration.

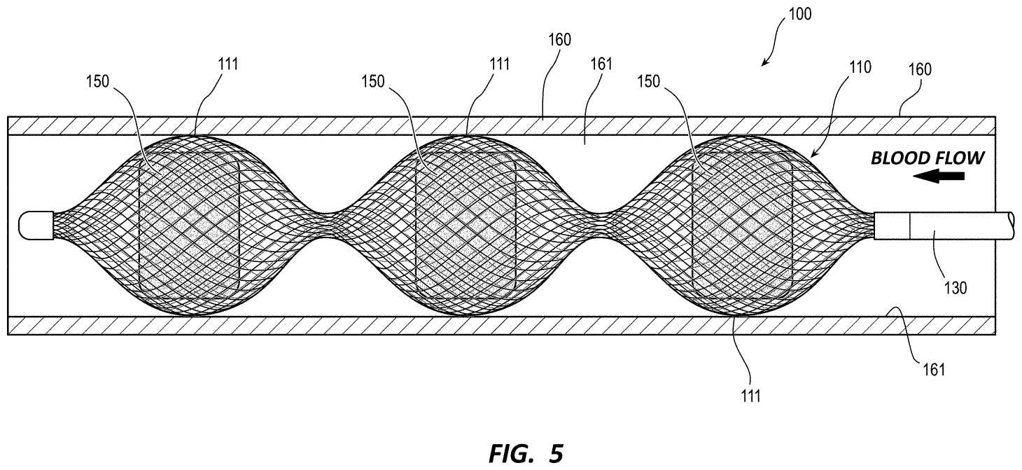

When deployed within a blood vessel 160, the embolic structure 110 and the embolic particles 150 can transition from the constrained state to a partially expanded state, such as shown in FIG. 5. In some embodiments, the enclosures 111 may self-expand when disposed outside the delivery catheter until the embolic structure 110 contacts a vessel wall 161. The placement wire 130 may be decoupled from the embolic structure 110, for example by rotating the placement wire 130 relative to the embolic structure 110 to release the placement wire 130 from the embolic structure 110. FIG. 5 depicts a deployed six mm embolic structure 110 within a four mm blood vessel 160. As shown, the enclosures 111 of the embolic structure 110 are partially radially expanded and the embolic particles 150 are disposed within the enclosures 111.

When deployed, the embolic structure 110 and the embolic particles 150 can form a mechanical blood flow restrictor within the blood vessel 160. The embolic particle 150 size, density of wires 113 of the enclosures 111, embolic particle 150 material, degree of expansion of the enclosures 111, and other parameters may affect the degree to which flow across the embolization device 100 is restricted by the embolization device 100. Embodiments wherein blood flow is reduced from about 10% to about 50% or more are within the scope of this disclosure. In some embodiments, the density or matrix of the wires 113 is configured to allow blood flow in the enclosures 111 and to prevent the embolic particles 150 from escaping from the enclosures 111. In other words, the spacing of the wires 113 may be wide enough to allow blood flow into the enclosures 111 but small enough to prevent the embolic particles 150 from passing between the wires 113.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified. For example, a method of manufacturing an embolization device may include one or more of the following steps: braiding a plurality of wires to form an enclosure of an embolic structure; disposing an embolic particle into a hub of a needle; coupling a fluid dispensing device to the hub of the needle; applying pressure to a fluid within the fluid dispensing device; and injecting the embolic particle into the enclosure, wherein the enclosure is in a partially expanded state.

References to approximations are made throughout this specification, such as by use of the term “about.” For each such reference, it is to be understood that, in some embodiments, the value, feature, or characteristic may be specified without approximation. For example, where a qualifier such as “about” is used, this term includes within its scope the qualified words in the absence of its qualifiers. For example, where the term “about” is recited with respect to a feature, it is understood that in further embodiments, the feature can have a precise configuration.

Similarly, in the above description of embodiments, various features are sometimes grouped together in a single embodiment, figure, or description thereof for the purpose of streamlining the disclosure. This method of disclosure, however, is not to be interpreted as reflecting an intention that any claim require more features than those expressly recited in that claim. Rather, as the following claims reflect, inventive aspects lie in a combination of fewer than all features of any single foregoing disclosed embodiment.

The claims following this written disclosure are hereby expressly incorporated into the present written disclosure, with each claim standing on its own as a separate embodiment. This disclosure includes all permutations of the independent claims with their dependent claims. Moreover, additional embodiments capable of derivation from the independent and dependent claims that follow are also expressly incorporated into the present written description.

Without further elaboration, it is believed that one skilled in the art can use the preceding description to utilize the invention to its fullest extent. The claims and embodiments disclosed herein are to be construed as merely illustrative and exemplary, and not a limitation of the scope of the present disclosure in any way. It will be apparent to those having ordinary skill in the art, with the aid of the present disclosure, that changes may be made to the details of the above-described embodiments without departing from the underlying principles of the disclosure herein. In other words, various modifications and improvements of the embodiments specifically disclosed in the description above are within the scope of the appended claims. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified. The scope of the invention is therefore defined by the following claims and their equivalents.

1. An embolization device, comprising:
an embolic structure comprising a self-expanding enclosure, wherein the self-expanding enclosure comprises a braided lattice of wires; and
an embolic particle disposed within the self-expanding enclosure.

2. The embolization device of claim 1, wherein the embolic particle comprises a gelatin material.

3. The embolization device of claim 2, wherein the gelatin material is one or more of collagen and polyvinyl alcohol.

4. The embolization device of claim 2, wherein the gelatin material is bioabsorbable.

5. The embolization device of claim 1, wherein the embolic particle includes a defined shape when dry and an amorphous shape when hydrated.

6. The embolization device of claim 5, wherein the defined shape is any one of a cube, cylinder, and ball.

7. The embolization device of claim 1, wherein the embolic particle is in a form of a matrix or foam and comprises pores.

8. The embolization device of claim 1, wherein the embolic particle comprises a thrombogenic agent configured to promote thrombus formation adjacent the embolic particle.

9. The embolization device of claim 1, wherein the wires comprise a shape memory material.

10. The embolization device of claim 1, wherein the wires comprise nitinol.

11. The embolization device of claim 1, wherein the embolic structure further comprises a plurality of self-expanding enclosures and a necked down middle portion disposed between the plurality of self-expanding enclosures.

12. The embolization device of claim 1, wherein the embolic structure further comprises a first clamp coupled to a first end and a threaded clamp coupled to a second end.

13. The embolization device of claim 12, further comprising a placement wire selectively threadingly coupled to the threaded clamp.

14. A method of restricting blood flow within a blood vessel, comprising deploying an embolization device into a blood vessel, wherein the embolization device comprises an embolic structure comprising a self-expanding enclosure, and wherein the self-expanding enclosure contains an embolic particle.

15. The method of claim 14, wherein deploying the embolization device comprises deploying the embolic structure and the embolic particle into the blood vessel together.

16. The method of claim 14, further comprising forming a thrombus within the embolic structure adjacent the embolic particle.

17. The method of claim 14, further comprising restricting blood flow within the blood vessel from 10% to 50%.

18. A method of manufacturing an embolization device, comprising:
braiding a plurality of wires to form an enclosure of an embolic structure;
disposing an embolic particle into a hub of a needle;
coupling a fluid dispensing device to the hub of the needle;
pressurizing a fluid within the fluid dispensing device; and
injecting the embolic particle into the enclosure, wherein the enclosure is in a partially expanded state.

19. The method of claim 18, further comprising hydrating the embolic particle within the hub of the needle, wherein the embolic particle is viscous.

20. The method of claim 18, further comprising drying the embolic particle disposed within the enclosure.
